(19) United States
(12) Patent Application Publication
(10) Pub. No.: US 2026/0279531 A1
(43) Pub. Date: Sep. 17, 2026

(54) ELECTRONIC HEALTH RECORD GENERATION SYSTEMS

(71) Applicant: ScribeAmerica, LLC, Fort Lauderdale, FL (US)

(72) Inventor: Vadim Vitalyevich Khazan, Seattle, WA (US)

(21) Appl. No.: 19/565,307

(22) Filed: Mar. 12, 2026

Related U.S. Application Data
(60) Provisional application No. 63/771,305, filed on Mar. 13, 2025.

(51) Int. Cl. G16H 15/00; G10L 15/26; G16H 10/60

(52) U.S. Cl. CPC G16H 15/00; G10L 15/26; G16H 10/60

(57) ABSTRACT

Some aspects include of a computer system or a computer-implemented method for generating a medical chart using a machine learning model. The performance of the machine learning model can be improved by updating the conditioning of the model with updated conditioning instructions. The machine learning model can be improved via the conditioning instructions to output improved charts that are tailored to the user's preferences and requirements, thereby enhancing the efficiency and accuracy of medical documentation. The systems and methods can allow the provider to submit feedback and receive an updated chart incorporating the feedback in real-time (e.g., in response to providing the feedback).

This application claims the benefit of U.S. Provisional Application Ser. No. 63/771,305, filed Mar. 13, 2025. The disclosure of the prior application is considered part of the disclosure of this application, and is incorporated in its entirety into this application.

### TECHNICAL FIELD

This specification generally relates to technological improvements to a computer platform for generating a medical chart, for example, using health care provider feedback for a session between a patient and the health care provider.

### BACKGROUND

When conducting a session with a patient, a health care provider typically asks the patient various questions in order to understand the patient's condition for purposes of achieving a diagnosis or improved outcome. For example, the health care provider can inquire about the patient's medical history, the nature of current symptoms the patient is experiencing, medications currently being taken by the patient, and so forth. After examining the patient and arriving at a diagnosis, the health care provider can formulate a plan for treating the patient, which may include various therapies and medical prescriptions. A medical chart that documents the session can be managed and stored by an electronic health record (EHR) system.

### SUMMARY

This document generally describes technological improvements to computer systems and computer-implemented methods for generating a medical chart using provider feedback. In some examples, medical charts are generated using an adaptive machine learning model that iteratively improves through user feedback. For example, the adaptive machine learning model can be implemented to interact with an EHR system and to update the conditioning of the machine learning model according to the feedback. The systems and methods disclosed herein are used to generate medical charts with inputs from a provider who is responsible for and provides the medical expertise and judgment.

In some implementations, a system for improved medical chart generation is disclosed. The system can include a computing system that is configured to electronically receive encounter data from a practitioner interface device. The encounter data can include a provider ID for a provider of a health care session and an audio file capturing audio data for the health care session. Responsive to receiving the encounter data, the computing system can be configured to computationally process the audio data to identify patterns of frequencies in the audio data and constructing a transcript based on the identified patterns of frequencies in the audio data and automatically and computationally construct first conditioning instructions for a machine learning model based on the provider ID. The computing system can be further configured to provide the transcript to a machine learning model conditioned by the first conditioning instructions to automatically and computationally output a first medical chart based on the transcript and the first conditioning instructions. The computing system can be configured to electronically transmit the first medical chart to the practitioner interface device. The practitioner interface device can be configured to present the first medical chart on a user interface and receive inputs providing feedback on the first medical chart from the provider. The computing system can be configured to receive the feedback on the first medical chart from the practitioner interface device. The computing system can be further configured to automatically and computationally tune the first conditioning instructions for the machine learning model to generate second conditioning instructions for the machine learning model based on the feedback on the first medical chart. The computing system can be configured to provide the transcript to the machine learning model conditioned by the second conditioning instructions to automatically and computationally output a second medical chart based on the transcript and the second conditioning instructions. The computing system can electronically transmit the second medical chart to the practitioner interface device. The practitioner interface device can be configured to present the second medical chart on the user interface and receive inputs providing a selection of whether the first medical chart or the second medical chart is preferred by the provider. The computing device can be configured to electronically receive the selection from the practitioner interface device. In response to receiving the selection, the computing system can be configured to store the first conditioning input for use in subsequent medical chart generation tasks for the provider and electronically transmit the first medical chart to an electronic health record system when the selection indicates that the first medical chart is preferred by the provider or store the second conditioning input for use in subsequent medical chart generation tasks for the provider and electronically transmit the second medical chart to the electronic health record system when the selection indicates that the second medical chart is preferred by the provider.

In some implementations, a computer-implemented method may be performed. The method can include receiving, at a computer system, encounter data of a health care session conducted by a health care provider; constructing, by the computer system, first conditioning instructions for a machine learning model; generating, by the computer system, a first chart for the health care session by providing the encounter data as input to the machine learning model conditioned by the first conditioning instructions to output the first chart; and receiving, at the computer system, feedback on the first chart. Responsive to receiving the feedback on the first chart the method can include automatically and computationally tuning, by the computer system, the first conditioning instructions for the machine learning model to generate second conditioning instructions for the machine learning model based on the feedback on the first chart; and automatically and computationally generating, by the computer system, a second chart for the health care session incorporating the feedback on the first chart by providing the encounter data to the machine learning model conditioned by the second conditioning instructions to output the second chart. In some examples, this process continues to further refine the conditioning instructions according to the user's feedback.

Other implementations of this aspect can include corresponding computer systems, and include corresponding apparatus and computer programs recorded on one or more computer storage devices, each configured to perform the actions of the method.

In another aspect, a computer system is disclosed. The computer system can include one or more computers and one or more computer memory devices interoperably coupled with the one or more computers and having tangible, non-transitory, machine-readable media storing one or more instructions. The instructions, when executed by the one or more computers, can cause the one computer system to perform one or more operations. These operations can include receiving encounter data of a health care session conducted by a health care provider; constructing first conditioning instructions for a machine learning model; generating a first chart for the health care session by providing the encounter data as input to the machine learning model conditioned by the first conditioning instructions to output the first chart; and receiving feedback on the first chart. Responsive to receiving the feedback on the first chart the instructions can further cause the computer system to automatically and computationally tune the first conditioning instructions for the machine learning model to generate second conditioning instructions for the machine learning model based on the feedback on the first chart; and automatically and computationally generate a second chart for the health care session by providing the encounter data to the machine learning model conditioned by the second conditioning instructions to output the second chart.

These and other implementations may optionally include any or all of the following features. The method and/or operations can further include storing, by the computer system, the second conditioning instructions, receiving, at the computer system, second encounter data of a second healthcare session conducted by the health care provider, retrieving, by the computer system, the second conditioning instructions, and generating, by the computer system, a third chart for the second healthcare session by providing the second encounter data to the machine learning model conditioned by the second conditioning instructions to output the third chart. The feedback can be received as one or more inputs at a user interface presenting the first chart. The user interface can provide a text box to receive the one or more inputs for the feedback. The user interface can present a selected list of feedback options to receive the one or more inputs for the feedback. The user interface can provide a chat interface to receive the one or more inputs for the feedback. The second conditioning instructions can be generated using machine learning. The first conditioning instructions can be generated, at least in part, according to a classification of the health care session, the health care provider, or both. The machine learning model can be a large language model. The second chart can be provided to an electronic health records system. The first chart can be provided to an electronic health records system. The encounter data can be generated by receiving, by the computer system, audio data capturing the health care session, processing, by the computer system, the audio data to identify patterns of frequencies in the audio data, and generating, by the computer system, the encounter data based on the identified patterns of frequencies in the audio data. The encounter data can include a transcript of the health care session. The second chart can be a patient chart. The method and/or operations can further include providing, by the computer system, the first chart and the second chart to a user interface, receiving, by the computer system, an input indicating whether the first chart or the second chart is preferred, storing, by the computer system, the second conditioning instructions when the input indicates that the second chart is preferred, and storing, by the computer system, the first conditioning instructions when the input indicates that the first chart is preferred. The method and/or operations can further include tuning, by the computer system, the first conditioning instructions to generate third conditioning instructions when the input indicates that the first chart is preferred. tuning the first conditioning instructions to generate the second conditioning instructions are further based on tuning instructions. The method and/or operations can further include receiving, by the computer system, additional feedback on the second chart, tuning, by the computer system, the second conditioning instructions based on the additional feedback on the second chart to generate an third conditioning instructions, and generating, by the computer system, a third chart for the health care session by providing the encounter data to the machine learning model conditioned by the third conditioning instructions to output the third chart. The computer system can manage a database storing a plurality of conditioning instructions tuned and associated with a plurality of healthcare providers. The computer system manages a database storing a plurality of conditioning instructions for the health care provider each associated with a different type of healthcare session.

Operations and method steps performed automatically and computationally include processes that use a computing system including software and hardware to complete the process by the computing system. For example, the computing system completes the method step and/or operation on its own in response to receiving a set of inputs and/or instructions that triggers the method steps or operations.

The systems, devices, program products, and processes described throughout this document can, in some instances, provide one or more of the following advantages. The performance of the machine learning model is improved by updating the conditioning of the model with updated conditioning instructions. For example, the machine learning model is improved via the conditioning instructions to output improved charts that are tailored to the user's preferences and requirements, thereby enhancing the efficiency and accuracy of medical documentation. The techniques disclosed herein allows the provider to submit feedback and receive an updated chart incorporating the feedback in real-time (e.g., in response to providing the feedback). The techniques disclosed herein further allow the provider to customize the charts according to their specific use case. The systems and methods disclosed herein are used to generate medical charts with inputs from a practitioner who is responsible for and provides the medical expertise and judgment. The systems and methods can include mechanisms and user interface elements which ensure that the provider reviews and makes final approval of the information in the medical chart. The outputs from the example machine learning models are not intended to substitute for human medical expertise or judgment. In the preferred embodiments, the example machine learning models are useful for transforming inputs from a provider (which include the medical expertise and judgment among other information) into a formatted medical chart and presents the generated medical chart to the provider for confirmation and final approval. When a health care provider ends a session recording, a practitioner interface device can automatically begin transferring an audio file to a system server using resumable upload techniques, such that a file transfer can resume from a last point of transfer (rather than from the beginning), if a transfer is interrupted at any point due to a poor network connection. After an audio file has been successfully uploaded, the uploaded file can be deleted from local storage of the practitioner interface device, thus freeing up storage resources, while complying with legal and organizational data retention requirements. By trimming and noise filtering uploaded audio files, downstream processes can be more efficiently and accurately performed, and data storage can be conserved.

Other features, aspects and potential advantages will be apparent from the accompanying description and figures.

### DESCRIPTION OF DRAWINGS

FIG. 1 is a diagram of an example system for generating a medical chart with provider feedback using a machine learning model.

FIG. 2 is a diagram that illustrates example technology and workflow features for generating a medical chart.

FIG. 3 illustrates an example system flow diagram for generating a medical chart using the machine learning model.

FIG. 4 illustrates an example system flow diagram for generating updated conditioning instructions used to update the machine learning model to generate a medical chart that incorporates the provider feedback.

FIG. 5 is a lane diagram illustrating an example method for generating a medical chart with provider feedback.

FIG. 6 illustrates an example user interface for receiving inputs providing the provider feedback.

FIG. 7 illustrates an example user interface for receiving inputs providing the provider feedback.

FIG. 8 illustrates an example user interface for receiving a selection for a user's preferred health report.

FIG. 9 is a schematic diagram that shows an example of a computing device and a mobile computing device.

Like reference symbols in the various drawings indicate like elements.

### DETAILED DESCRIPTION

This document generally describes computer systems, processes, program products, and devices for generating a medical chart using provider feedback. In some examples, medical charts are generated using an adaptive machine learning model that iteratively improves through user feedback. For example, the adaptive machine learning model can be implemented to interact with an electronic health record (EHR) system and to update the conditioning of the machine learning model according to the feedback. The example machine learning models disclosed herein can be used to generate medical charts with inputs from a provider who is ultimately responsible for and provides the medical expertise and judgment. The example machine learning models disclosed herein do not substitute for medical expertise or judgment.

Some examples include systems and methods with an adaptive medical chart generation features. The systems or methods can use a machine learning model and user feedback to update a medical chart according to feedback from a user. The machine learning model can be a large language model. The system assists in the creation of medical documentation by processing transcripts with the machine learning model that is conditioned according to initial conditioning instructions to produce a first chart. Users (e.g., the provider) can provide feedback through one or more user interfaces, including direct chat inputs and predefined checkboxes addressing aspects such as verbosity and structure of the medical chart. This feedback is used to refine the conditioning instructions used to condition the machine learning model to enhance the quality and relevance of the generated charts.

In some examples, the process involves an iterative cycle where an initial chart is generated using the machine learning model conditioned with initial conditioning instructions. User feedback and the initial conditioning instructions are then used to create updated conditioning instructions. The machine learning model, conditioned with the updated conditioning instructions, processes the transcript to produce an updated chart. The user can review the updated chart (e.g., to compare the initial chart and the updated chart), and if the updated chart is deemed superior by the user, the new conditioning instructions are retained for future use. Otherwise, the system can revert to the original conditioning instructions and can repeat the process or iteratively refine the conditioning instructions. Advantages of this adaptive mechanism includes continuous improvement of the machine learning model. In some examples, the improvements to the machine learning model provide improvements in chart generation, tailored to user preferences and requirements, thereby enhancing the efficiency and accuracy of medical documentation.

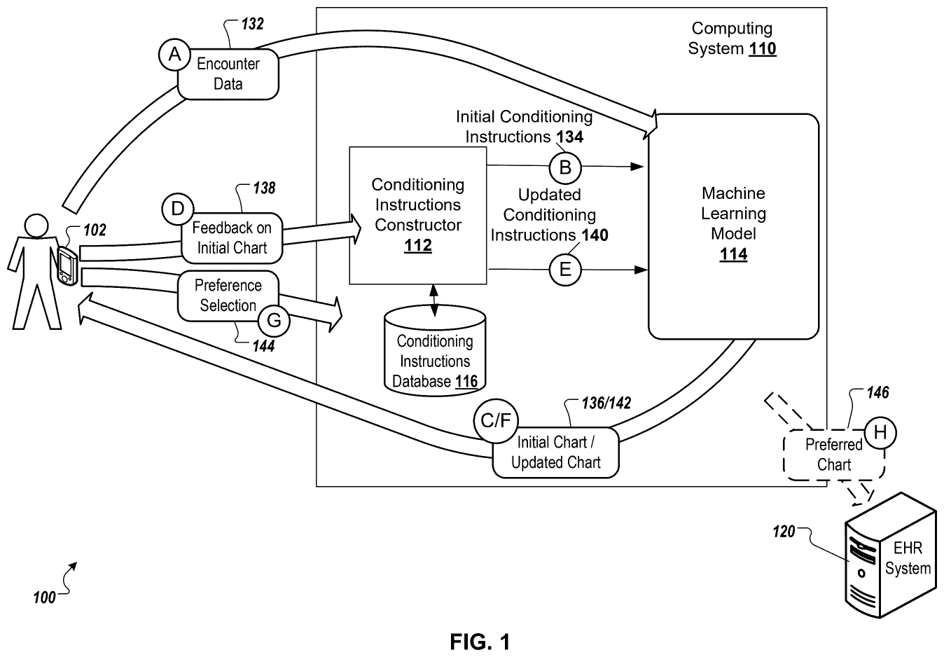

FIG. 1 is a diagram of an example system 100 (e.g., an electronic health record generation system) for generating a medical chart with user (e.g., a provider) feedback using a machine learning model 114, as represented in example stages (A) to (H). Stages (A) to (H), for example, may occur in the illustrated sequence, a different sequence, and/or two or more stages (A) to (H) may be concurrent. In some examples, one or more stages (A) to (H) may be repeated multiple times when generating a medical chart.

In the present example, the system 110 includes a practitioner interface device 102. The practitioner interface device 102 can include personal computers, laptop computers, smartphones, digital assistants, tablets, or other sorts of stationary or mobile computing devices that are configured to receive input from an operator (e.g., tactile input received through a controls presented on a touch screen and/or physical device controls, spoken input received through a microphone, activation commands received from a remote control device (such as a Bluetooth device), etc.), to present output to the operator (e.g., tactile, audio, and/or visual interfaces and notifications), to record a health care session conducted by the operator (e.g., with the recording including audio data, audiovisual data, etc.), and to communicate with other system components over communications network(s) (not shown). For example, the practitioner interface device 102 can be configured to communicate with the computing system 110 and/or the electronic health record (EHR) system 120 via one or more communication networks. The communication network(s), for example, can include one or more of a LAN (local area network), a WAN (wide area network), and/or the Internet.

The computing system 110 can include one or more computing device (e.g., computing servers such as application servers, data servers, cloud servers, etc.). The computing system 110 is configured to electronically communicate with the practitioner interface device 102 and the EHR system 120 via the one or more communication networks. In some implementations, an application programming interface (API) of the computing system 110 can use web sockets to send data to and receive data from the practitioner interface device 102 and the EHR system 120 in real time. For example, many features of the API can be shared by practitioner interface applications running on the practitioner interface device 102. In the example shown, the computing system includes a conditioning instructions constructor 112, a machine learning model 114, and a conditioning instructions database 116.

The conditioning instructions constructor 112 is configured to generate conditioning instructions used to condition the machine learning model 114. In some examples, the conditioning instructions constructor retrieves the conditioning instructions from the conditioning instructions database 116.

The machine learning model 114 is configured to receive encounter data 132 (e.g., a transcript and meta data) from a health care session and generate a chart for the healthcare session. In some examples, the machine learning model 114 is a large language model. In some examples, the machine learning model 114 is trained on example data that includes example charts and example transcripts associated with the example charts. The machine learning model 114 is configured to be conditioned according to conditioning instructions. The conditioning instructions can be constructed for a particular provider, practice, type of encounter, or other characteristics of a health care encounter.

The conditioning instructions database 116 stores conditioning instructions for a plurality of providers. In some examples, each provider may have multiple conditioning instructions associated with different types of patient encounters and stored at the conditioning instructions database 116. The conditioning instructions database 116 can include data servers, file systems, and/or other suitable types of data storage devices or systems. The conditioning instructions database 116 can manage and store a plurality of conditioning instructions tuned and associated with a plurality of healthcare providers. In some examples, the conditioning instructions database 116 can manage and store a plurality of conditioning instructions for the health care provider each associated with a different type of healthcare session.

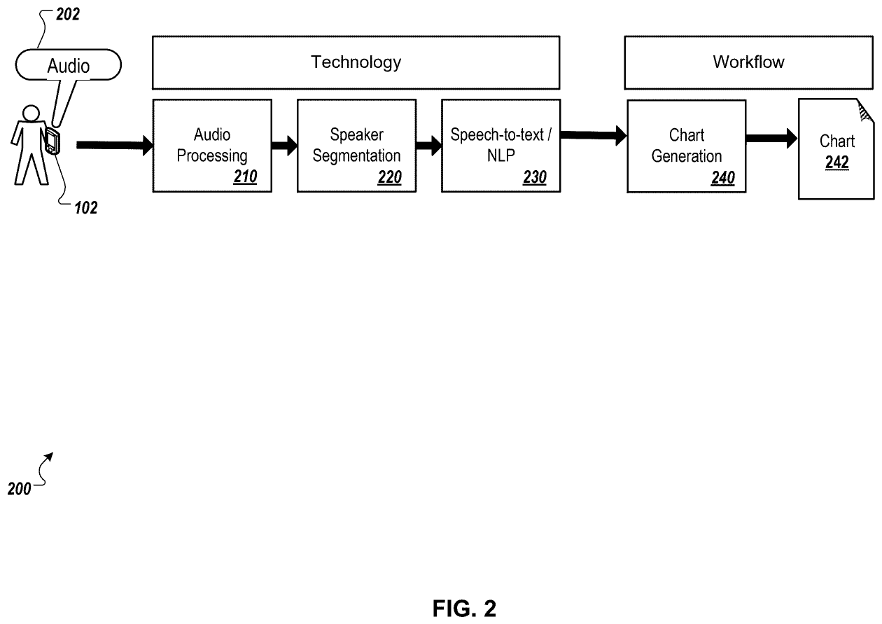

During stage (A), the computing system 110 receives encounter data 132 from the practitioner interface device 102. In some examples, the encounter data 132 includes a transcript of a healthcare session between the user (e.g., the provider/practitioner) and the patient. In some examples, the encounter data includes an audio file that is processed by the computing system 110 to generate the transcript. For example, as described in FIG. 2.

During stage (B), the conditioning instructions constructor 112 generates initial conditioning instructions 134. In some examples, the encounter data 132 includes a provider ID and/or data a classification of the encounter (e.g., an encounter type ID). The conditioning instructions constructor 112 uses this information to retrieve the appropriate portions of conditioning instructions from the conditioning instructions database 116 and compiles the initial conditioning instructions 134 with these portions. In some implementations, for example, when the user is new to the system, default conditioning instructions are used as the initial conditioning instructions 134. The default conditioning instructions can be conditioning instructions tuned for primary care. In some examples, complete conditioning instructions for the user and/or the type of encounter is stored at the conditioning instructions database 116 and is retrieved for use as the initial conditioning instructions 134.

In some examples, the encounter data 132 includes metadata or a tag that identifies the provider (e.g., a provider ID). In response to the computing system 110 receiving the encounter data 132, The Conditioning instructions constructor 112 can identify the provider ID in the encounter data and use the provider ID to retrieve conditioning instructions from the conditioning instructions database 116. The conditioning instructions can include several instructions defining different sections of a medical chart. The conditioning instructors constructor 112 can compile the instructions for the appropriate sections for a particular provider and/or type of encounter (e.g., some users may have several different types of visits and want different characteristics in the medical charts depending on the type of visit). In some examples, the conditioning instructions constructor 112 will inject instructions that reflect a providers preferences. In some examples, a user can modify the sections or the provider preferences through a user interface that presents a template for the medical chart.

The machine learning model 114 is conditioned with the initial conditioning instructions 134 and processes the encounter data 132 (e.g., the transcript) to generate an initial chart 136. The initial chart 136 is provided to the practitioner interface device 102 at stage (C).

During stage (C) the user can view the initial chart 136, via a user interface at the practitioner interface device 102, to approve the chart and/or to provide feedback via inputs to the practitioner interface device 102. For example, the user can provide inputs indicating that they would prefer certain sections of the medical chart include full sentences or to use bullet points. As another example, the user may provide feedback that they want the medical chart to include more or less information. Many other examples of feedback can be provided by the user at the practitioner interface device 102.

In some examples, the user may be satisfied with the initial chart 136 and may provide inputs indicating that the initial chart is approved. In this instance, the practitioner interface device 102 can provide the approval message to the computing system 110 which may store the initial conditioning instructions 134 in the conditioning instructions database 116 and/or provide the initial chart 136 to the EHR system 120 (e.g., at stage H).

During stage (D) the feedback on the initial chart 148 is provided to the conditioning instructions constructor 112. The conditioning instructions constructor 112 uses the feedback on the initial chart 148 to tune the initial conditioning instructions 134 to generate the updated conditioning instructions 140.

During stage (E) the machine learning model 114 is conditioned with the updated conditioning instructions. The machine learning model receives and process the encounter data 132 (e.g., the transcript) to generate the updated chart 142. The computing system 110 provides the updated chart to the practitioner interface device 102 at stage (F).

During stage (F), the computing system 110 provides the updated chart 142 to the practitioner interface device 102. The practitioner interface device 102 generates a user interface which allows the user to review the updated chart. In the example shown, the user provides inputs for a preference selection 144 indicating whether the updated chart or the initial chart is preferred. In some examples, the user can provide additional feedback on the updated chart 142 and the process repeats to further tune the conditioning instructions based on the user's feedback.

During stage (G), the preference selection 144 is provided to the computing system 110. The computing system 110 can be configured to store the conditioning instructions associated with the preferred chart in the conditioning instructions database 116. For example, if the user selects the initial chart 136 as the preferred chart, then the initial conditioning instructions 134 can be stored in the conditioning instructions database 116 and will be used for subsequent encounters uploaded by the user. If the user selects the updated chart 142 as the preferred chart, then the updated conditioning instructions 140 can be stored in the conditioning instructions database 116 and will be used for subsequent encounters uploaded by the user.

During stage (H) the computing system 110 provides the preferred chart 146 to the EHR system 120. For example, the computing system 110 may interface with an API for the EHR system 120 which allows the computing system 110 to provide a medical chart for the patient to the EHR system 120.

As discussed, the stages may be iteratively performed. For example, a user may provide feedback on charts at any time while reviewing charts for approval. In response to the feedback the system 100 can be configured to perform stages D, E, F, and G to update the chart in accordance with the user's feedback.

FIG. 2 is a diagram that illustrates example technology and workflow features 200 for generating a medical chart. As shown in FIG. 2, for example, practitioner interface device 102 (e.g., the practitioner interface device 102 shown in FIG. 1) can be used to record audio data 202 during or after a health care session between a health care provider and a patient (e.g., one or more audio files included in the encounter data 132, shown in FIG. 1). In some examples, the audio data 202 can be or can include a lossless, uncompressed audio file (e.g., 16,000 Hz), or another audio format that is suitable for speech-to-text operations.

The audio data 202 can be provided to an audio processing engine 210, which can perform basic checks to ensure that the audio data is correctly formatted and does not include computer viruses. After performing the basic checks, for example, the audio processing engine 210 can perform noise filtering and selective silence reduction on the audio data 202. A selective silence reduction operation, for example, can include detecting voice activity in the audio data (e.g., based on the frequency of human voices), and eliminating portions of the audio data in which speaking does not occur. A trimmed audio data can be run through a noise filtering operation to minimize background noise that may be present in the audio. By trimming and noise filtering the audio data 202, for example, downstream processes can be more efficiently and accurately performed (e.g., improving a word error rate in an automatically generated transcript), and data storage can be conserved.

Processed audio data 202 can be provided to a speaker segmentation engine 220, which can identify segments of the audio according to the speaker. For example, the speaker segmentation engine 220 can provide the processed audio data 202 to a machine learning model that is trained to recognize segments of the audio that are spoken by a particular health care provider, and segments that are spoken by other individuals. Training the machine learning model to recognize audio segments of the particular health care provider, for example, can occur as part of an onboarding process for the health care provider. For example, audio data of the health care provider speaking can be collected during the onboarding process to acquire an acoustic model of the health care provider's voice, which can be used to improve the performance of the machine learning model during production. As another example, audio data of an initial encounter conducted by the health care provider can be manually segmented, labeled, and provided to the machine learning model for training, and processed audio data for subsequent encounters can be provided to the machine learning model for automatic segmentation.

The processed and segmented audio data 202 can be provided to a speech-to-text/natural language processing (NLP) engine 230, which can generate a transcript for the session between the health care provider and the patient, with each segment of spoken audio being labeled by speaker.

The audio processing engine 210, the speaker segmentation engine 220, and the speech-to-text/NLP engine 230, for example, can each include software and/or hardware components of the computing system 110 (shown in FIG. 1). After the process for processing the encounter data 132 (shown in FIG. 1) is complete, metadata associated with the encounter data 132, the generated transcript, one or more audio files corresponding to the processed audio data 202 (and optionally, the original audio data) can be stored by the computing system 110, e.g., at an encounter data store.

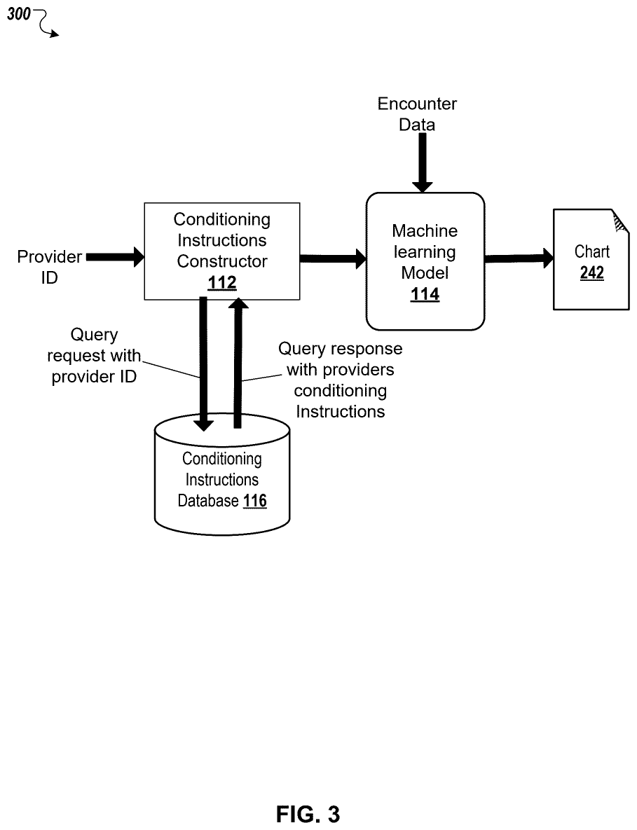

The encounter data is provided to the chart generation engine 240 which processes the encounter data to generate a medical chart 242. For example, using the process illustrated in FIG. 3 or other techniques disclosed herein.

The technology can provide one or more of the following advantages. When a health care provider ends a session recording, a practitioner interface 102 device can automatically begin transferring an audio data 202 to the computer system using resumable upload techniques, such that a file transfer can resume from a last point of transfer (rather than from the beginning), if a transfer is interrupted at any point due to a poor network connection. After an audio file has been successfully uploaded, the uploaded file can be deleted from local storage of the practitioner interface device 102, thus freeing up storage resources. By trimming and noise filtering uploaded audio files, downstream processes can be more efficiently and accurately performed, and data storage can be conserved. The technology can also ensure that the data is maintained, stored, moved, secured, and managed in a manner that is compliant with legal regulations and organizational policies (e.g., data retention policies) as well as industry best practices.

FIG. 3 is a system flow diagram illustrating a process 300 for generating a medical chart 242 using the machine learning model 114. The example shown includes a conditioning instructions constructor 112, a machine learning model 114, and a conditioning instructions database 116, each of which is illustrated and described in reference to FIG. 1. The conditioning instructions constructor 112 receives a provider ID (which may be included as part of the encounter data or derived from the metadata) for the provider requesting the medical chart 242. The conditioning instructions constructor 112 sends a query request with the provider ID to the conditioning instructions database 116 to retrieve conditioning instructions associated with the provider. In some examples, the conditioning instructions are constructed based on various parameters that can include default parameters as well as parameters selected by the provider (e.g., to identify desired sections of a medical chart to include or required characteristics for the chart). The conditioning instructions are used to condition the machine learning model 114. The machine learning model 114 is conditioned according to the conditioning instructions and processes the encounter data (e.g., the generated transcript described in reference to FIG. 2) to output the medical chart 242. In some examples, the conditioning instructions and encounter data are combined by the conditioning instructions constructor and provided as an input to the machine learning model 114 (e.g., as a prompt for an LLM).

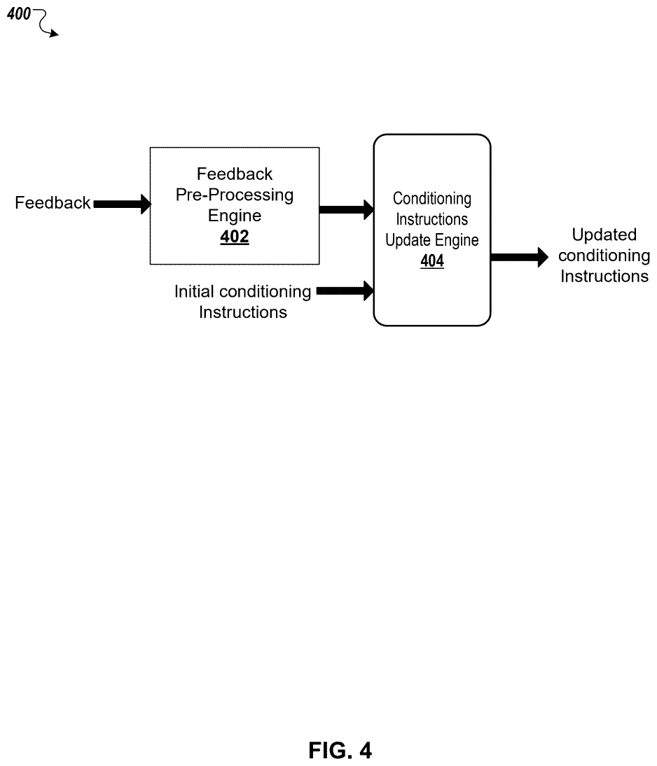

FIG. 4 is an example system flow diagram illustrating a method 400 for generating updated conditioning instructions used to update the machine learning model to generate a medical chart that incorporates the user's (e.g., the provider's) feedback. As discussed in FIG. 1, the machine learning model can be updated with updated conditioning instructions based on feedback received from the user. The feedback can be provided to a feedback pre-processing engine 402. and the feedback may be provided directly to the conditioning instructions update engine. The feedback pre-processing engine 402 can structure the feedback. For example, the feedback pre-processing engine 402 may label the feedback to identify a specific portion of the medical chart where the feedback should be applied or the feedback can be applied with a label that indicates that it should be applied to the entire chart. In some examples, the feedback pre-processing engine 402 processes the instructions to make sure that the feedback is valid and implementing the feedback would not violate any required parameters of a medical chart (e.g., as defined by the provider and/or the EHR provider).

The structured feedback and the initial feedback is provided to the conditioning instructions update engine 404 which tunes the initial conditioning instructions in accordance with the structured feedback. In some examples, the conditioning instructions update engine 404 includes a machine learning model that is trained to tune the initial conditioning instructions in accordance with the feedback. In some examples, the machine learning model is a large language model that is instructed (e.g., via tuning instructions) to process the feedback and initial conditioning instructions to generate updated conditioning instructions. In some examples, the machine learning model is the same machine learning model used to generate the medical charts. Some implementations do not include the feedback-pre-processing engine. Some implementations do not use machine learning to tune the conditioning instructions.

In some examples, the feedback pre-processing engine 402 includes a classifier which identifies one or more sections of the medical chart where the feedback is relevant. The conditioning instructions engine 404 can then identify conditioning instructions for the one or more sections and tune the instructions for the one or more sections based on the feedback. In some examples, the classifier may identify that the feedback is relevant for the entire medical chart and the conditioning instructions engine 404 can modify a conditioning instructions direct to the entire chart based on the feedback. For example, the user may submit feedback to make the entire chart more concise, the classifier will determine that the feedback is directed to the entire chart and the conditioning instructions engine 404 can tune a header instruction that conditions the machine learning model to generate a more concise medical chart.

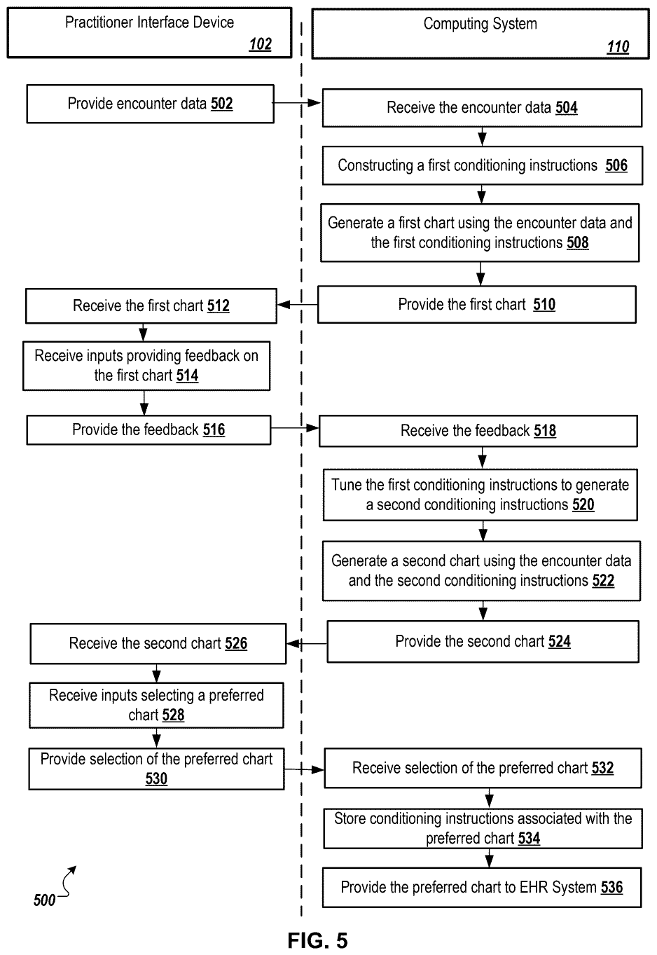

FIG. 5 is a lane diagram illustrating an example method 500 for generating a medical chart with user (e.g., provider) feedback. In the present example, the method 500 can be performed by the computing system 110 (shown in FIG. 1), and a practitioner interface device 102 (e.g., also shown in FIG. 1). The method 500 includes operations 502-536.

At the operation 502, the practitioner interface device 102 provides the encounter data to the computing system 110. At the operating 504, the computing system 110 receives the encounter data from the practitioner interface device 102. In some examples, the encounter data includes a transcript of a healthcare session between a provider and a patient. The transcript can be generated at the practitioner interface device 102 or the computing system 110 in different implementations. In some examples, the transcript is generated by processing the audio data to identify patterns of frequencies in the audio data and generating the transcript based on the identified patterns of frequencies in the audio data (e.g., using a speech-to-text model).

At the operation 506, the computing system 110 constructs first conditioning instructions for a machine learning model. In some examples, the first conditioning instructions are default conditioning instructions. For example, the first conditioning instructions can be a default for a type of encounter. In other examples, the first conditioning instructions are the current conditioning instructions associated with the provider.

At the operation 508, the computing system generates a first chart (e.g., a patient medical chart) using the encounter data of the healthcare session and the first conditioning instructions. In some examples, the first chart is generated by providing the encounter data as input to the machine learning model conditioned by the first conditioning instructions to output the first chart. In some examples, the machine learning model is trained to output a medical chart. In some examples, the machine learning model is an LLM. In some examples, the first conditioning instructions are generated, at least in part, according to a classification of the health care session, the health care provider, or both.

At the operation 510, the computing system 110 provides the first chart to the practitioner interface device 102. At the operation 512, the practitioner interface device 102 receives the first chart from the computing system 110. The practitioner interface device 102 presents the initial chart via a user interface to receive approval of the initial chart and/or feedback on the initial chart.

At the operation 514, the practitioner interface device 102 receives inputs providing feedback on the first chart. The feedback can be received as one or more inputs at the user interface presenting the first chart. In some examples, the user interface provides a text box to receive the one or more inputs for the feedback. In some examples, the user interface presents a selectable list of feedback options to receive the one or more inputs for the feedback. In some examples, the user interface provides a chat interface to receive the one or more inputs for the feedback.

At the operation 516, and responsive to receiving the feedback on the first chart, the practitioner interface device 102 provides the feedback to the computing system 110. At the operation 518, the computing system 110 receives the feedback from the practitioner interface device 102.

At the operation 520, the computing system 110 tunes the first conditioning instructions to generate second conditioning instructions for the machine learning model based on the feedback on the first chart. In some examples, the second conditioning instructions are generated using machine learning. In some examples, the same LLM used to generate the chart is used to tune the first conditioning instructions to generate the second conditioning instructions. In some examples, the conditioning instructions are tuned using tuning instructions (e.g., as an additional input to the LLM).

At the operation 522, the computing system 110 generates a second chart (e.g., a patient medical chart) using the encounter data and the conditioning instructions. The second chart can be generated by providing the encounter data to the machine learning model conditioned by the second conditioning instructions to output the second chart.

At the operation 524, the computing system 110 provides the second chart to the practitioner interface device 102. At the operation 526, the practitioner interface device 102 receives the second chart from the computing system 110.

At the operation 528, the practitioner interface device 102 receives inputs selecting a preferred chart. For example, the user can review the second chart and determine if the second chart appropriately incorporated the user feedback and, if so, select an input indicating the second chart is preferred.

At the operation 530, the practitioner interface device 102 provides the selection of the preferred chart to the computing system 110. At the operation 532, the computing system 110 receives a selection of the preferred chart.

At the operation 534, the computing system 110 stores the conditioning instructions associated with the preferred chart. For example, if the second chart is selected as the preferred chart then the second conditioning instructions are stored and if the user provides inputs rejecting the update chart then the first conditioning instructions are stored.

At the operation 536, the computing system 110 provides the preferred chart to an EHR system. For example, the computing system can provide the preferred chart for the patient to the EHR system 120 (shown in FIG. 1). The EHR system can store the chart with the patient's records.

The stored conditioning instructions can be used for the user in subsequent encounters. For example, the method can further include to receive, at the computer system 110, second encounter data of a second healthcare session conducted by the health care provider, retrieve the stored conditioning instructions for the health care provider, and generate, by the computer system, a third chart for the second healthcare session by providing the second encounter data to the machine learning model conditioned by the retrieved conditioning instructions to output the third chart.

In some examples, the method can be performed iteratively to update the conditioning instructions as the user provides more feedback at the practitioner interface device 102. For example, the method can further include operations to receive, by the computer system 110, additional feedback on the second chart (e.g., captured via inputs at the practitioner interface device 102), tune, by the computer system 110, the second conditioning instructions based on the additional feedback on the second chart to generate third conditioning instructions, and generate, by the computer system 110, a third chart for the health care session by providing the encounter data to the machine learning model conditioned by the third conditioning instructions to output the third chart.

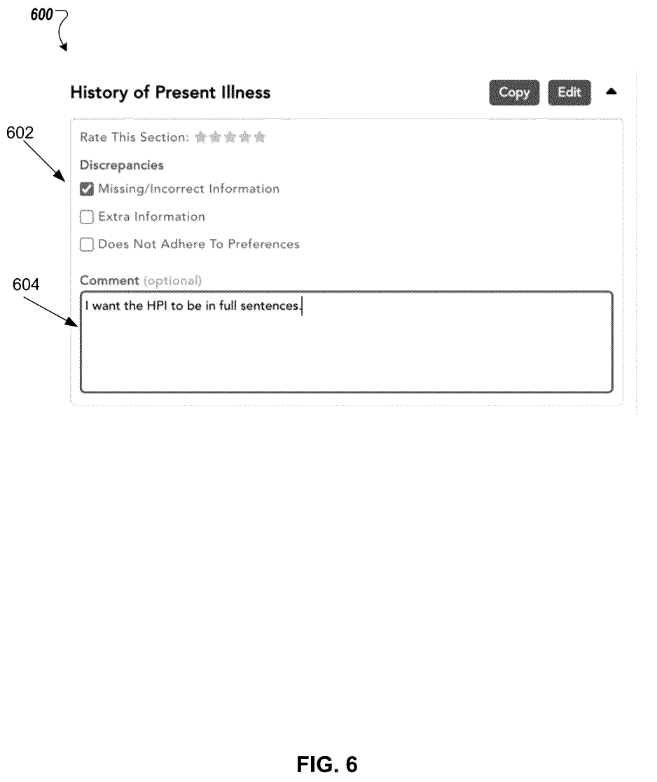

FIG. 6 illustrates an example user interface 600 for receiving inputs providing the user (e.g., provider) feedback. In some examples, the user interface 700 is presented at the practitioner interface device 102 (shown in FIG. 1) to receive feedback on a chart from the user. In some examples, the user interface 600 is presented (e.g., a pop-up) while the user reviews different sections of the medical chart. In the example shown, the user can provide feedback on a specific section of the medical chart by selecting a selectable list of common feedback options at 602. The user interface 600 also includes a text box to receive feedback at 604.

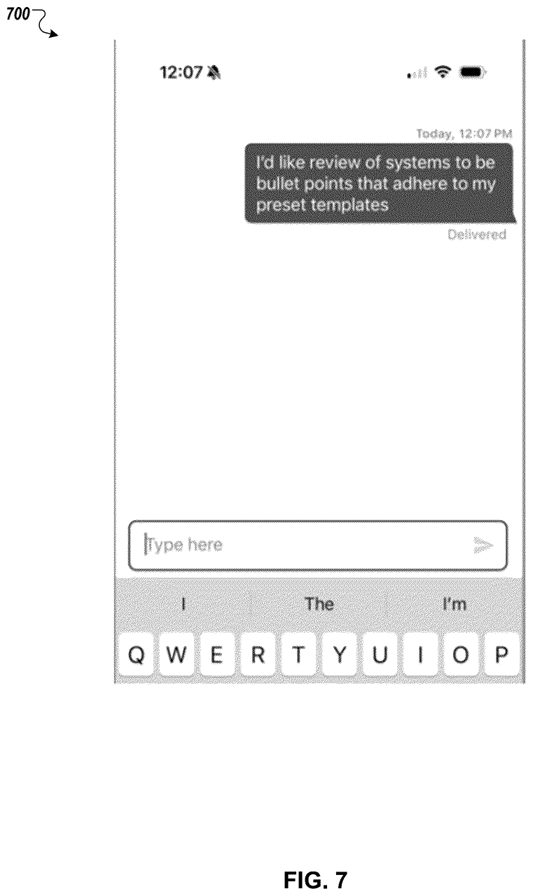

FIG. 7 illustrates an example user interface 700 for receiving inputs providing the user (e.g., provider) feedback. In some examples, the user interface 700 is presented at the practitioner interface device 102 (shown in FIG. 1) to receive feedback on a chart from the user. In the example shown, the user interface includes a chat interface. In some examples, the chart interface uses a large language model to receive feedback from the user. The chat interface allows a provider to submit free form feedback, which can be processed by the computing system (e.g., at the conditioning instructions update engine 404 shown in FIG. 4) to tune the conditioning instructions.

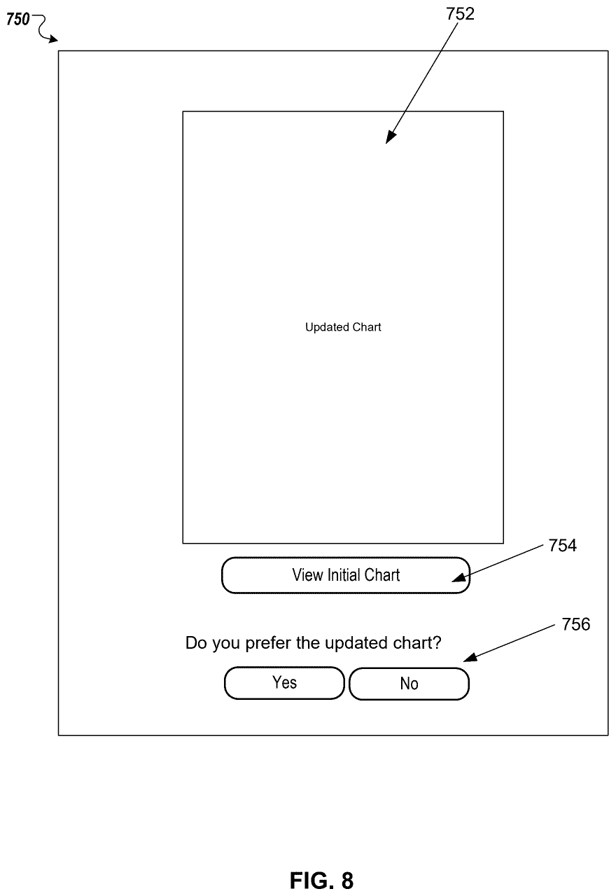

FIG. 8 illustrates an example user interface 750 for receiving a selection for a user's preferred health report. In some examples, the user interface 750 is presented at the practitioner interface device 102 (shown in FIG. 1) to receive a selection from a user identifying whether the user prefers the updated chart or an initial chart. The user interface 750 presents the updated chart at 752 to allow the practitioner to review the update chart. The user interface 750 includes a button 754, which when selected presents the initial chart at 752. This allows the user to compare the initial chart and the updated chart before selecting a preferred chart. In alternative interfaces, both the updated chart and initial chart can be presented at the same time (e.g., side by side). The user interface 750 is configured to receive a selection at 756 identifying whether the user prefers the updated chart or an initial chart.

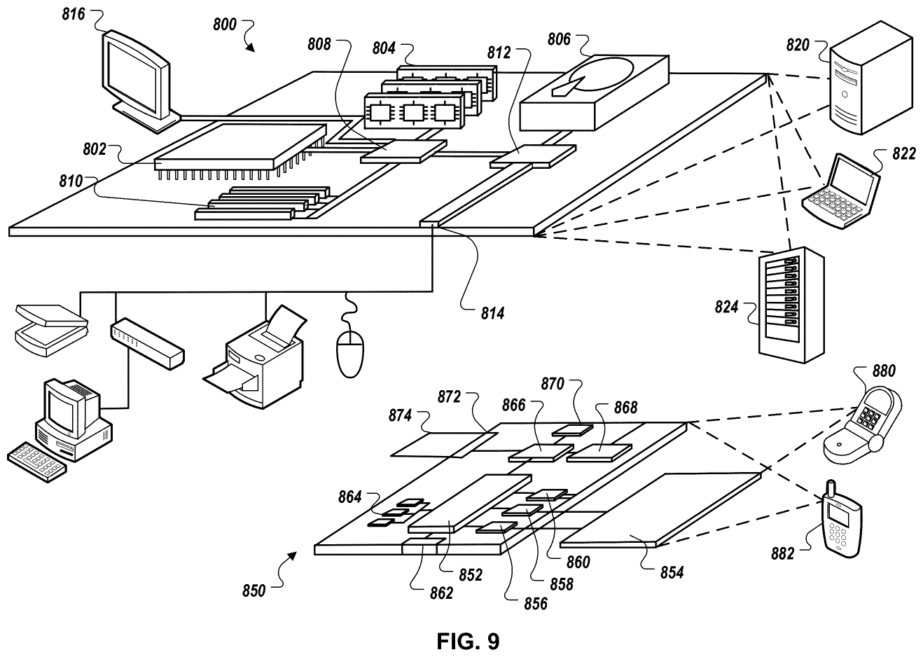

FIG. 9 shows an example of a computing device 800 and an example of a mobile computing device 850 that can be used to implement the techniques described here. The computing device 800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and can be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 804 stores information within the computing device 800. For example, the memory 804 is a volatile memory unit or units. For example, the memory 804 is a non-volatile memory unit or units. The memory 804 can also be another form of computer-readable medium, such as a magnetic or optical disk.

The storage device 806 is capable of providing mass storage for the computing device 800. For example, the storage device 806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer-or machine-readable medium, such as the memory 804, the storage device 806, or memory on the processor 802.

The high-speed interface 808 manages bandwidth-intensive operations for the computing device 800, while the low-speed interface 812 manages lower bandwidth-intensive operations. Such allocation of functions is exemplary only. For example, the high-speed interface 808 is coupled to the memory 804, the display 816 (e.g., through a graphics processor or accelerator), and to the high-speed expansion ports 810, which can accept various expansion cards (not shown). In the implementation, the low-speed interface 812 is coupled to the storage device 806 and the low-speed expansion port 814. The low-speed expansion port 814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 800 can be implemented in a number of different forms, as shown in the figure. For example, it can be implemented as a standard server 820, or multiple times in a group of such servers. In addition, it can be implemented in a personal computer such as a laptop computer 822. It can also be implemented as part of a rack server system 824. Alternatively, components from the computing device 800 can be combined with other components in a mobile device, such as mobile computing device 850. Each of such devices can contain one or more of the computing device 800 and the mobile computing device 850, and an entire system can be made up of multiple computing devices communicating with each other.

The mobile computing device 850 includes a processor 852, a memory 864, an input/output device such as a display 854, a communication interface 866, and a transceiver 868, among other components. The mobile computing device 850 can also be provided with a storage device, such as a micro-drive or other device, to provide additional storage. Each of the processor 852, the memory 864, the display 854, the communication interface 866, and the transceiver 868, are interconnected using various buses, and several of the components can be mounted on a common motherboard or in other manners as appropriate.

The processor 852 can execute instructions within the mobile computing device 850, including instructions stored in the memory 864. The processor 852 can be implemented as a chipset of chips that include separate and multiple analog and digital processors. The processor 852 can provide, for example, for coordination of the other components of the mobile computing device 850, such as control of user interfaces, applications run by the mobile computing device 850, and wireless communication by the mobile computing device 850.

The processor 852 can communicate with a user through a control interface 858 and a display interface 856 coupled to the display 854. The display 854 can be, for example, a TFT (Thin-Film-Transistor Liquid Crystal Display) display or an OLED (Organic Light Emitting Diode) display, or other appropriate display technology. The display interface 856 can comprise appropriate circuitry for driving the display 854 to present graphical and other information to a user. The control interface 858 can receive commands from a user and convert them for submission to the processor 852. In addition, an external interface 862 can provide communication with the processor 852, so as to enable near area communication of the mobile computing device 850 with other devices. The external interface 862 can provide, for example, for wired communication For example, or for wireless communication in other implementations, and multiple interfaces can also be used.

The memory 864 stores information within the mobile computing device 850. The memory 864 can be implemented as one or more of a computer-readable medium or media, a volatile memory unit or units, or a non-volatile memory unit or units. An expansion memory 874 can also be provided and connected to the mobile computing device 850 through an expansion interface 872, which can include, for example, a SIMM (Single In Line Memory Module) card interface. The expansion memory 874 can provide extra storage space for the mobile computing device 850, or can also store applications or other information for the mobile computing device 850. Specifically, the expansion memory 874 can include instructions to carry out or supplement the processes described above, and can include secure information also. Thus, for example, the expansion memory 874 can be provided as a security module for the mobile computing device 850, and can be programmed with instructions that permit secure use of the mobile computing device 850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. For example, a computer program product is tangibly embodied in an information carrier. The computer program product contains instructions that, when executed, perform one or more methods, such as those described above. The computer program product can be a computer-or machine-readable medium, such as the memory 864, the expansion memory 874, or memory on the processor 852. For example, the computer program product can be received in a propagated signal, for example, over the transceiver 868 or the external interface 862.

The mobile computing device 850 can communicate wirelessly through the communication interface 866, which can include digital signal processing circuitry where necessary. The communication interface 866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 868 using a radio-frequency. In addition, short-range communication can occur, such as using a Bluetooth, WiFi, or other such transceiver (not shown). In addition, a GPS (Global Positioning System) receiver module 870 can provide additional navigation-and location-related wireless data to the mobile computing device 850, which can be used as appropriate by applications running on the mobile computing device 850.

The mobile computing device 850 can also communicate audibly using an audio codec 860, which can receive spoken information from a user and convert it to usable digital information. The audio codec 860 can likewise generate audible sound for a user, such as through a speaker, e.g., in a handset of the mobile computing device 850. Such sound can include sound from voice telephone calls, can include recorded sound (e.g., voice messages, music files, etc.) and can also include sound generated by applications operating on the mobile computing device 850.

The mobile computing device 850 can be implemented in a number of different forms, as shown in the figure. For example, it can be implemented as a cellular telephone 880. It can also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. A system for improved medical chart generation, the system comprising:
a computing system configured to:
electronically receive encounter data from a practitioner interface device, wherein the encounter data includes a provider ID for a provider of a health care session and an audio file capturing audio data for the health care session;
responsive to receiving the encounter data, identify patterns of frequencies in the audio data and constructing a transcript based on the identified patterns of frequencies in the audio data;
automatically and computationally construct first conditioning instructions for a machine learning model based on the provider ID;
provide the transcript to a machine learning model conditioned by the first conditioning instructions to automatically and computationally output a first medical chart based on the transcript and the first conditioning instructions;
electronically transmit the first medical chart to the practitioner interface device, wherein the practitioner interface device is configured to present the first medical chart on a user interface and receive inputs providing feedback on the first medical chart from the provider;
receive the feedback on the first medical chart from the practitioner interface device;
automatically and computationally tune the first conditioning instructions for the machine learning model to generate second conditioning instructions for the machine learning model based on the feedback on the first medical chart;
provide the transcript to the machine learning model conditioned by the second conditioning instructions to automatically and computationally output a second medical chart based on the transcript and the second conditioning instructions;
electronically transmit the second medical chart to the practitioner interface device, wherein the practitioner interface device is configured to present the second medical chart on the user interface and receive inputs providing a selection of whether the first medical chart or the second medical chart is preferred by the provider;
electronically receive the selection from the practitioner interface device; and
in response to receiving the selection:
store the first conditioning input for use in subsequent medical chart generation tasks for the provider and electronically transmit the first medical chart to an electronic health record system when the selection indicates that the first medical chart is preferred by the provider; or
store the second conditioning input for use in subsequent medical chart generation tasks for the provider and electronically transmit the second medical chart to the electronic health record system when the selection indicates that the second medical chart is preferred by the provider.

2. A computer-implemented method comprising:
receiving, at a computer system, encounter data of a health care session conducted by a health care provider;
constructing, by the computer system, first conditioning instructions for a machine learning model;
generating, by the computer system, a first chart for the health care session by providing the encounter data as input to the machine learning model conditioned by the first conditioning instructions to output the first chart;
receiving, at the computer system, feedback on the first chart;
responsive to receiving the feedback on the first chart:
automatically and computationally tuning, by the computer system, the first conditioning instructions for the machine learning model to generate second conditioning instructions for the machine learning model based on the feedback on the first chart; and
automatically and computationally generating, by the computer system, a second chart for the health care session incorporating the feedback on the first chart by providing the encounter data to the machine learning model conditioned by the second conditioning instructions to output the second chart.

3. The computer-implemented method of claim 2, the method further comprising:
storing, by the computer system, the second conditioning instructions;
receiving, at the computer system, second encounter data of a second healthcare session conducted by the health care provider;
retrieving, by the computer system, the second conditioning instructions; and
generating, by the computer system, a third chart for the second healthcare session by providing the second encounter data to the machine learning model conditioned by the second conditioning instructions to output the third chart.

4. The computer-implemented method of claim 2, wherein the feedback is received as one or more inputs at a user interface presenting the first chart.

5. The computer-implemented method of claim 4, wherein the user interface provides a text box to receive the one or more inputs for the feedback.

6. The computer-implemented method of claim 4, wherein the user interface presents a selectable list of feedback options to receive the one or more inputs for the feedback.

7. The computer-implemented method of claim 4, wherein the user interface provides a chat interface to receive the one or more inputs for the feedback.

8. The computer-implemented method of claim 2, wherein the second conditioning instructions are generated using machine learning.

9. The computer-implemented method of claim 2, wherein the first conditioning instructions are generated, at least in part, according to a classification of:
(i) the health care session,
(ii) the health care provider, or
both (i) and (ii).

10. The computer-implemented method of claim 2, wherein the machine learning model is a large language model.

11. The computer-implemented method of claim 2, wherein the second chart is provided to an electronic health records system.

12. The computer-implemented method of claim 2, wherein the encounter data is generated by:
receiving, by the computer system, audio data capturing the health care session;
processing, by the computer system, the audio data to identify patterns of frequencies in the audio data; and
generating, by the computer system, the encounter data based on the identified patterns of frequencies in the audio data.

13. The computer-implemented method of claim 2, wherein the encounter data includes a transcript of the health care session.

14. The computer-implemented method of claim 2, wherein the second chart is a patient medical chart.

15. The computer-implemented method of claim 2, the method further comprising:
providing, by the computer system, the first chart and the second chart to a user interface;
receiving, by the computer system, an input indicating whether the first chart or the second chart is preferred;
storing, by the computer system, the second conditioning instructions when the input indicates that the second chart is preferred; and
storing, by the computer system, the first conditioning instructions when the input indicates that the first chart is preferred.

16. The computer-implemented method of claim 15, wherein the method includes:
tuning, by the computer system, the first conditioning instructions to generate third conditioning instructions when the input indicates that the first chart is preferred.

17. The computer-implemented method of claim 2, wherein tuning the first conditioning instructions to generate the second conditioning instructions are further based on tuning instructions.

18. The computer-implemented method of claim 2, the method further comprising:
receiving, by the computer system, additional feedback on the second chart;
tuning, by the computer system, the second conditioning instructions based on the additional feedback on the second chart to generate third conditioning instructions; and
generating, by the computer system, a third chart for the health care session by providing the encounter data to the machine learning model conditioned by the third conditioning instructions to output the third chart.

19. A computer system comprising:
one or more computers; and
one or more computer memory devices interoperably coupled with the one or more computers and having tangible, non-transitory, machine-readable media storing one or more instructions that, when executed by the one or more computers, perform one or more operations comprising:
receiving encounter data of a health care session conducted by a health care provider;
constructing first conditioning instructions for a machine learning model;
generating a first chart for the health care session by providing the encounter data as input to the machine learning model conditioned by the first conditioning instructions to output the first chart;
receiving feedback on the first chart;
responsive to receiving the feedback on the first chart:
automatically and computationally tuning the first conditioning instructions for the machine learning model to generate second conditioning instructions for the machine learning model based on the feedback on the first chart; and
automatically and computationally generating a second chart for the health care session by providing the encounter data to the machine learning model conditioned by the second conditioning instructions to output the second chart.

20. The computer system of claim 19, wherein the computer system manages a database storing a plurality of conditioning instructions tunned and associated with a plurality of healthcare providers.

21. The computer system of claim 19, wherein the computer system manages a database storing a plurality of conditioning instructions for the health care provider each associated with a different type of healthcare session.
